(19) United States
(12) Patent Application Publication
(10) Pub. No.: US 2005/0019381 A1
(43) Pub. Date: Jan. 27, 2005

(54) Binding agent which is stable in storage and used for pharmaceutical applications

(76) Inventors: Hans-Ulrich Petereit, Darmstadt (DE); Manfred Assmus, Bickenbach (DE); Thomas Beckert, Warthausen (DE); Guenther Bergmann, Gross-Krotzenburg (DE); Stephanie Zacharias, Bensheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/239,187

(22) PCT Filed: Jan. 29, 2001
(86) PCT No.: PCT/EP01/00923

(51) Int. Cl. A61K 9/70

(52) U.S. Cl. 424/449

(57) ABSTRACT

The invention relates to an adhesive and binder for dermal or transdermal therapy systems, consisting of (a) a (meth)acrylate copolymer composed of radically polymerized C₁ to C₄ alkyl esters of acrylic or methacrylic acid and (meth)acrylate monomers with a cationic ammonium group in the alkyl radical, containing (b) 0.1-45 wt.-% with reference to (a) of an organic dicarboxylic or tricarboxylic acid or an acrylate or (meth)acrylate polymer or copolymer containing an acid group, as well as (c) 20-80 wt.-% with reference to (a) of a plasticizer, and (d) if necessary, a pharmaceutically active substance and/or pharmaceutically usual additives, characterized in that dibutyl sebacate is contained as the plasticizer.

The invention relates to an adhesive and binder for pharmaceutical use that is stable during storage.

### STATE OF THE ART

WO 00/33821 describes medications made up of a core, containing pravastatin and a coating that dissolves in gastric juices, made of a (meth)acrylate copolymer, which can contain a hydrophobic plasticizer, if necessary, such as dibutyl sebacate. In this connection, the formulations are supposed to be free of polyanionic polymers or acids with a low molecular weight.

EP-A 848 950 and EP-A 848 960 describe adhesives and binders for pharmaceutical purposes that are characterized by a high level of hydrophilia, i.e., a high permeability for water vapor, and, at the same time, demonstrate a high adhesive force at low cold flow. The adhesives and binders are therefore very well suited as skin adhesives or transdermal therapy systems.

EP-A 0 848 960 describes an adhesive and binder for dermal or transdermal therapy systems consisting of (a1) 55-99.9 wt.-% of a (meth)acrylate copolymer made of structural and functional monomers, the functional monomers containing tertiary or quaternary amino groups, (a2) 0.1-45 wt.-0% of an acrylate or (meth)acrylate polymer or copolymer containing an acid group, and (b) 25-80 wt.-%, with reference to the total of (a1) and (a2), of a plasticizer. A large number of suitable plasticizers, including diethyl sebacate, is listed. However, the problems of storage stability are not mentioned.

EP-A 848 950 describes an adhesive and binder for dermal or transdermal therapy systems, consisting of (a) 85-99 wt.-% of a (meth)acrylate copolymer made up of structural and functional monomers, the functional monomers having tertiary or quaternary amino groups, (b) 15-0.1 wt.-% of an organic dicarboxylic or tricarboxylic acid, as well as (c) 40-70 wt.-%, with reference to the total of (a) and (b), of a plasticizer. A large number of suitable plasticizers, including diethyl sebacate, is listed. However, the problems of storage stability are not mentioned.

### TASK AND SOLUTION

One problem of adhesives and binders that contain plasticizers, particularly those that are based on (meth)acrylate copolymers, is plasticizer migration or decomposition during extended periods of storage. As a result, the original content of plasticizer can escape, and therefore, of course, the properties of such forms of medications change in an unforeseeable and undesirable manner.

As it has turned out, this problem occurs in particularly disruptive manner in comparatively hydrophilic adhesives and binders, particularly also those that are known from EP-A 848 950 and EP-A 848 960.

A task of the present invention was to develop improved adhesives and binders for pharmaceutical preparations that are characterized by good storage stability, without their other positive properties being impaired.

Surprisingly, it was found that this task is accomplished by an adhesive and binder for dermal or transdermal therapy systems, consisting of

(a) a (meth)acrylate copolymer composed of radically polymerized C₁ to C₄ alkyl esters of acrylic or methacrylic acid and (meth)acrylate monomers with a cationic ammonium group in the alkyl radical, containing

(b) 0.1-45 wt.-% with reference to (a) of an organic dicarboxylic or tricarboxylic acid or an acrylate or (meth)acrylate polymer or copolymer containing an acid group, as well as

(c) 20-80 wt.-% with reference to (a) of a plasticizer, and

(d) if necessary, a pharmaceutically active substance and/or pharmaceutically usual additives,

characterized in that diethyl sebacate is contained as the plasticizer (c).

This task is also accomplished by the use of diethy sebacate and/or dibutyl sebacate as the plasticizer (c) in an adhesive and binder for dermal or transdermal therapy systems consisting of

(a) a (meth)acrylate copolymer composed of radically polymerized C₁ to C₄ alkyl esters of acrylic or methacrylic acid and (meth)acrylate monomers with a cationic ammonium group in the alkyl radical, containing

(b) 0.1-45 wt.-% with reference to (a) of an organic dicarboxylic or tricarboxylic acid or an acrylate or (meth)acrylate polymer or copolymer containing an acid group,

(c) 25-80 wt.-% of a plasticizer, with reference to (a),

(d) if necessary, a pharmaceutically active substance and/or pharmaceutically usual additives,

for the purpose of obtaining a formulation that demonstrates a residual content of at least 90% of the plasticizer originally used, after storage for 6 months at 40° C. and 75% relative humidity.

It was not foreseeable that the stated tasks could be accomplished by the special selection of the plasticizer(s) diethy sebacate and/or dibutyl sebacate.

### EMBODIMENT OF THE INVENTION

Component (a)

Component (a) is a (meth)acrylate copolymer composed of radically polymerized C₁ to C₄ alkyl esters of acrylic or methacrylic acid and (meth)acrylate monomers with a cationic ammonium group in the alkyl radical. This definition includes copolymers that have been known for a long time as drug coatings, under the product names EUDRAGIT® E, EUDRAGIT® RS, or EUDRAGIT® RL, among others.

C₁ to C₄ alkyl esters of acrylic or methacrylic acid are, in particular, methy* acrylate, ethy* acrylate, butyl acrylate, butyl methacrylate, and methyl methacrylate.

(Meth)acrylate monomers with a cationic ammonium group in the alkyl radical are, in particular, (meth)acrylate monomers with tertiary or quaternary amino or ammonium groups in the alkyl radical.

The content of the functional monomers with tertiary ammonium groups can advantageously be between 30 and 70 wt.-%, preferably between 40 and 60 wt.-%.

Suitable monomers with tertiary ammonium groups are listed in U.S. Pat. No. 4,705,695, column 3, line 64, to column 4, line 13. The following should be particularly mentioned: dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate, and diethylamino-2,2-dimethyl)propyl methacrylate. Dimethylaminoethyl methacrylate is particularly preferred.

A (meth)acrylate copolymer with tertiary amino groups, corresponding to component (a), can be composed, for example, of 20-30 wt.-% methyl methacrylate, 20-30 wt.-% butyl methacrylate, and 60-40 wt.-% dimethylaminoethyl methacrylate.

A (meth)acrylate copolymer with tertiary amino groups, corresponding to component (a), can be composed, for example, of 25 wt.-% methyl methacrylate, 25 wt.-% butyl methacrylate, and 50 wt.-% dimethylaminoethyl methacrylate (EUDRAGIT® E 100).

2-trimethylammonium ethyl methacrylate chloride is particularly preferred as a monomer with functional quaternary ammonium groups. The content of the functional monomers with quaternary ammonium groups preferably lies between 2 and 15 wt.-%.

Corresponding (meth)acrylate copolymers are known, for example, from EP-A 181 515 or DE-PS 1 617 751. These are soluble or swellable polymerizates, independent of pH, which are suitable as drug coatings. A possible production method that can be mentioned is substance polymerization in the presence of a radical-forming initiator dissolved in the monomer mixture. Likewise, the polymerizate can also be produced by means of solution or precipitation polymerization. In this manner, the polymerizate can be obtained in the form of a fine powder, which can be achieved, in the case of substance polymerization, by means of grinding, or, in the case of solution or precipitation polymerization, by spray drying, for example.

A corresponding copolymer can also be composed, for example, of 50-70 wt.-% methyl methacrylate, 20-40 wt.-% ethyl acrylate, and 7-2 wt.-% 2-trimethyl ammonium methyl methacrylate chloride.

Another suitable (meth)acrylate copolymer can be composed, for example, of 85 to less than 93 wt.-% C1 to C4 alkyl esters of acrylic or methacrylic acid, and more than 7 to 15 wt.-% (meth)acrylate monomers with a quaternary ammonium group in the alkyl radical. Such (meth)acrylate monomers are commercially available and have been used as time-release coatings for a long time.

A (meth)acrylate copolymer corresponding to component (a), with quaternary amino groups, can be composed, for example, of 60 wt.-% methyl methacrylate, 30 wt.-% ethyl acrylate, and 10 wt.-% 2-trimethyl ammonium methyl methacrylate chloride (EUDRAGIT® RL 100).

Another preferred (meth)acrylate copolymer corresponding to component (a), with quaternary amino groups, can be composed, for example, of 65 wt.-% methyl methacrylate, 30 wt.-% ethyl acrylate, and 5 wt.-% 2-trimethyl ammonium methyl methacrylate chloride (EUDRAGIT® RS 100).

The copolymers (a) are obtained in known manner, by means of radical substance, solution, bead, or emulsion polymerization. They can be present as extruded granulate, ground powder, solution, or dispersion.

Component (b)

Component (b) functions as the counter-ion for the cationic component (a). Component (b) can be adjusted in such a way that partial or almost complete neutralization of the tertiary in component (a) is brought about. Partial neutralization in the range of 2-50% is preferred.

Component (b) is composed of 0.1-45, preferably 1 to 30, particularly preferably 5 to 25 wt.-% with reference to (a) of an organic dicarboxylic or tricarboxylic acid or an acrylate or (meth)acrylate polymer or copolymer containing acid groups.

Accordingly, organic dicarboxylic, tricarboxylic acids are suitable, preferably succinic acid (succinate), fumaric acid, or citric acid.

Another suitable component (b) is acrylate or (meth)acrylate polymers or copolymers containing acid groups.

For example, polyacrylic acid (®Carbopol) is suitable.

However, copolymers of structural and functional (meth)acrylate monomers are preferred. Structural acrylate or methacrylate monomers are, for example, C₁ to C₄ alkyl esters of acrylic or methacrylic acid. Methy acrylate, ethy acrylate, butyl acrylate, and methyl methacrylate are preferred. Methacrylic acid is particularly preferred as a monomer with functional acid groups.

A copolymer corresponding to component (b) can be composed, for example, of 30-70 wt.-% ethyl acrylate or methyl methacrylate, and 70-30 wt.-% methacrylic acid.

It is essential for the present invention that components (a) and (b) are present in the ratios indicated. The proportion of component (a) is 55-99.9 wt.-%, and it is complemented by component (b) to total 100 wt.-%. If the proportion of the copolymer (b) that contains acid groups is less than 0.1 wt.-%, the adhesive strength is generally not sufficient. If the proportion is greater than 45 wt.-%, this has the disadvantage that the processability is impaired. The proportion of component (b) is preferably 15-0.1 wt.-%.

Component (c)

Component (c), a plasticizer, must be present in at least 25 and at most 80 wt.-%, preferably 30-60 wt.-%, with reference to component (a). At less than 25 wt.-% plasticizer, generally the skin adhesion that is achieved is insufficient. If the proportion is greater than 80 wt.-%, it is generally difficult to control the release of active substance.

Diethy sebacate and/or dibutyl sebacate are suitable as a plasticizer.

The addition of plasticizer allows adaptation of the physical properties to the requirements of the individual forms of medication, so that sufficient adhesion forces are achieved at room temperature or body temperature.

In addition, the plasticizers, at the ratios indicated, can advantageously lower the melt viscosity of the polymers used, in the liquid state. At room temperature, softening effects are evident.

Influences on the release behavior of embedded active substances are possible.

Component (d): Pharmaceutically Active Substances and/or Pharmaceutically Usual Additives

Pharmaceutically usual additives can be: Here, for example, stabilizers, dyes, anti-oxidants, wetting agents, pigments, lustring agents, etc. should be mentioned. They primarily serve as processing aids and are supposed to be able to guarantee a reliable and reproducible production process as well as good long-term storage capability. Other pharmaceutically usual processing aids can be present in amounts of 0.001 wt.-% to 100 wt.-%, preferably 0.1 to 50 wt.-% with reference to the copolymer. Examples of drying agents are: aluminum oxide, magnesium oxide, kaolin, talcum, silicic acid (aerosils), barium sulfate, carbon black, and cellulose. Examples of parting agents (mold parting agents) are: esters of fatty acids or fatty acid amides, aliphatic, long-chain carboxylic acids, fatty alcohols as well as their esters, mineral wax or paraffin wax and metal soaps; the following should be particularly mentioned: glycerin monostearate, stearyl alcohol, glycerin behenic acid ester, cetyl alcohol, palmitinic acid, carnauba wax, beeswax, etc.

Variations in the composition make it possible to balance out any undesirable effects of additives related to the form of medication. The adhesives and binders according to the invention can optionally contain other additives in small amounts, if special formulations require this: neutral polymers, tackifyers, stabilizers, dyes, anti-oxidants, wetting agents, pore formation agents, moisture retention agents, complex-forming agents, and others.

Production Method:

The production of the binder is dependent on the form of polymer used: Solid substances can be used directly, by mixing them with the additives in suitable mixers, kneaders, or extruders that can be heated and, if necessary, evacuated. The extruder is a single-screw or preferably a twin-screw extruder, in order to achieve suitable mixing and transport properties.

The processing temperature is guided by the melting properties of the materials, and preferably lies between 20° C. and 200° C. Limiting factors are the thermal stability of the substances used. Solid additives can be mixed with the polymer before extrusion. Liquid additives are added at about half the extrusion distance of the melt, and result in a reduction in viscosity and temperature.

Polymer solutions or dispersions are mixed with the additives, so that the latter are dissolved or suspended. The binder is obtained from these solutions, dispersions, or suspensions by means of drying to form thin film layers.

Processing:

Coating, granulation, sheathing or embedding take place using organic solutions or aqueous dispersions of suitable processing aids. The use of melts is limited to substances with defined melting points in the range of the processing temperatures. Usually, low melt viscosity values are needed for processing.

In a variant of the method, the solid adhesive and binder according to the invention is mixed with the powders and melted, either together or mixed with a suitable solvent.

Films, woven fabrics, or nonwoven fabrics are obtained by spreading the substances onto flat carriers, preferably from solution or suspension, or directly from the melt, and after drying or cooling, adhesive layers are obtained that fix the system in place on the skin and are particularly well tolerated because of their hydrophilia. Coating takes place in the laboratory, discontinuously, using a ductor blade, and on a technical scale or production scale, it takes place continuously, using a ductor roller or roller application. Immediately after coating, a slightly adhesive, often siliconized cover film is added, which is removed before use.

The agglomerates or adhesive layers obtained in this way can be processed further for use in forms of medications. In this connection, it is possible to work medicinal substances in as early as during production of the adhesive and binder. These active substances are then fixed in place in particular or dissolved form. It is possible to influence the release of active substance by means of the adhesive and binder, and this can be utilized for the formulation of forms of medications.

Forms of Medications

The medicinal substances used in the sense of the invention are intended for use in the human or animal body, in order to:

1. heal, relieve, prevent, or detect diseases, complaints, bodily injuries, or disease symptoms;

2. allow the composition, the status, or the functions of the body, or psychological states, to become evident;

3. replace active substances or bodily fluids produced by the human or animal body;

4. defend against disease pathogens, parasites, or substances foreign to the body, eliminate them, or make them harmless, or

5. influence the composition, the status, or the functions of the body, or psychological states.

Conventional medications can be found in reference works, such as the Rote Liste [Red List] or the Merck Index.

Any active substances that fulfill the desired therapeutic effect as defined above and possess sufficient thermal stability can be used according to the invention.

Important examples (groups and individual substances) are the following, without any claim to completeness:

Analgesics,

anti-allergy substances, anti-arrhythmics,

antibiotics, chemotherapy substances, antidiabetics, antidotes,

anti-epileptics, antihypertensives, antihypotensives,

anticoagulants, antimycotics, antiphlogistics,

beta receptor blockers, calcium antagonists, and ACE inhibitors,

broncholytics/anti-asthmatics, cholinergics, corticoids (internal),

dermatics, diuretics, enzyme inhibitors, enzyme preparations and transport proteins,

expectorants, geriatrics, gout medications, flu medications,

hormones and their inhibitors, hypnotics/sedatives, cardiac medications, lipid-lowering medications,

parathyroid hormones/calcium metabolism regulators,

psychoactive drugs, sexual hormones and their inhibitors,

spasmolytics, sympatholytics, sympathomimetics, vitamins,

wound treatment substances, cytostatics.

Examples of Active Substances are:

The invention is particularly suitable for making available forms of medications containing the following active substances:

Therapeutic Categories:

Analgesics, antirheumatics, anti-allergy substances, anti-arrhythmics, beta receptor blockers, calcium channel blockers, inhibitors of the renin/angiotensin system, broncholytics/anti-asthmatics, cholinergics, diuretics. Substances that promote circulation, gout medications, flu medications, coronary medications, lipid-lowering substances, gastrointestinal medications, psychoactive drugs, thrombocyte aggregation inhibitors, urologics, vein therapy medications, vitamins, and minerals.

Active Substances

Morphine and/or its derivatives, tramadol, acetylsalicylic acid, diclofenac, indometacin, lonazolac, ibuprofen, ketoprofen, propyphenazone, naproxen, paracetamol, flurbiprofen, dimetindene, quinidine, metoprolol, propranolol, oxprenolol, pindolol, atenolol, metoprolol [sic - repeated], disopyramide, verapamil, diltiazem, gallopamil, nifedipine, nicardipine, nisoldipine, nimodipine, amiodipine, theophylline, salbutamol, sildenafil, terbutaline, ambroxol, aminophylline, choline theophyllinate, pyridostigmine, piretanide, furosemide, pentoxyfylline, naftidrofuryl, buflomedil, xantinol nicotinate, bencyclane, allopurinol, norephedrine, chlorphenamine, isosorbide mononitrate, isosorbide dinitrate, glycerin trinitrate, molsidomine, bezafibrate, fenofibrate, gemfibrozil, cerivastatin, pravastatin, fluvastatin, lovastatin, atorvastatin, simvastatin, xantinol, metoclopramide, amitryptiline, dibenzepine, venlafaxin, thioridazine, oxazepam, lithium, nitrofurantoin, dried plant extract, ascorbic acid, and potassium and/or their salts used pharmaceutically.

Important active substances for transdermal therapy systems are, in particular: nicotine, glycerin trinitrate, scopolamine, clonidine, fentanyl, estradiol, testosterone, oxibutynine, diclophenac, deoxyribonucleic acids, for example for vaccines, ibuprofen, ketoprofen, diltiazem, propranolol, albuterol, alprazolam, amethocaine, atenolol, benzoporphyrin, buprenorphine, calcitonin, dithranol, diphencyprone, skin-penetrating peptides and peptides that are absorbed through the skin, eptazocine, ethinyl estradiol, methotrexate, or naloxon.

Forms of medication can be produced from the intermediate stages produced according to the invention, using conventional processing techniques.

Carriers with the adhesive and binder spread on them are generally present in the form of rolls, protected by cover films (release liners). Individual patches of the required size are cut or punched from these strips, and packaged individually.

Coating flat carriers with fluids that contain polymers is described, for example, in Mass, J. and Schmidt, H.: Coating Technology for Transdermal Drug Delivery Systems, Medical Device Technology, Edition 3/41990, p. 46-50.

Properties relevant for application, required tests, and specifications are listed in pharmacopoeias.

Details can be found in current reference works, such as:

Voigt, R. (1984): Lehrbuch der pharmazeutischen Technologie [Manual of pharmaceutical technology]; Verlag Chemie Weinheim—Beerfield Beach/Fla.—Basel.

Sucker, H., Fuchs, P., Speiser, P.: Pharmazeutische Technologie [Pharmaceutical technology], Georg Thieme Verlag Stuttgart (1991), particularly chapters 15 and 16, p. 626-642.

Gennaro, A. R. (editor), Remington's Pharmaceutical Sciences, Mack Publishing Co., Easton, Pa. (1985), Chapter 88, p.1567-1673.

Heilmann, K.: Therapeutische Systeme [Therapeutic systems], Ferdinand Euler Verlag, Stuttgart, p. 52-57.

Brandau, R., and Lippold, B. H. (1982): Dermal and Transdermal Absorption. Wissenschaftliche Verlagsgesellschaft mbH, Stuttgart, p.171-200.

### EXAMPLES

Please describe how the formulations of Examples 1 to 5 were produced and precisely how the measurements were taken.

### Example 1-5

The results of Examples 1 to 5 are summarized in Table 1:

Abbreviations for plasticizers: ATBC=acetyl tributyl citrate; TEC=triethyl citrate; DES=diethyl sebacate; DBS=dibutyl sebacate

EUDRAGIT® E 100: copolymer of 25 wt.-% methyl methacrylate, 25 wt.-% butyl methacrylate, and 50 wt.-% dimethylaminoethyl methacrylate.

TABLE 1
Plasticizer stability in EUDRAGIT ® E 100 adhesive formulations (Examples 1 to 3 are
comparison examples/Examples 4 and 5 are according to the invention

| | | | | | | Adhesive | |
|---|---|---|---|---|---|---|---|
| | | Storage | | % | | strength | |
| | | conditions | % | plasticizer | | values² | Tack³ |
| | | Time in months/ | plasticizer | analyzed | % | [N/10 mm] | 0-2 |
| | | temperature in | in | after | plasticizer | after | after |
| | | ° C./ | formulation | storage (% | of amount | production/ | production/ |
| | | rel. humidity | (% with ref. | with ref. | used, after | after | after |
| Ex. | Plasticizer/acid | in %¹ | to solid) | to solid) | storage | storage | storage |
| 1 | ATBC/ | 6/40/75 | 31.4 | 16.6 | 52.3 | 7.3/4.2 | 1/0 |
| | succinic acid | | | | | | |
| 2 | TEC/ | 3/40/75 | 24.7 | 14.3 | 57.9 | 6.5/0.3 | 1/0 |
| | succinic acid | | | | | | |
| 3 | TEC/ | 3/40/75 | 23.3 | 9.1 | 39.1 | not | 1/0 |
| | lactic acid | | | | | determined | |
| 4 | DES/ | 9/40/75 | 27.9 | 28.2 | 101 | 1.9/2.3 | 1/1 |
| | succinic acid | | | | | | |
| 5 | DBS/ | 9/40/75 | 27.9 | 30.5 | 109 | 2.8/2.4 | 1/1 |
| | succinic acid | | | | | | |

¹Packaging: heat-sealed sealed-edge bags (75 micrometers PE/12 micrometers PETP), i.e., air-tight seal.
²System of film/steel plate; 180° pull-off angle
³Determination of tack: a patch is pressed to the skin: 0 = no tack; 1 good tack after pressing down with gentle pressure; 2 = very good tack, no pressure required

1. A composition comprising
(a) a (meth)acrylate copolymer comprising radically polymerized C₁ to C₄ alkyl esters of acrylic or methacrylic acid and (meth)acrylate monomers with a cationic ammonium group in the alkyl radical,
(b) 0.1 to 45 wt. % of an organic dicarboxylic or tricarboxylic acid or an acrylate or (meth)acrylate polymer or copolymer comprising an acid group,
(c) 20 to 80 wt. % of a plasticizer, and
(d) optionally a pharmaceutically active substance, a pharmaceutically acceptable additive or mixture thereof,
wherein weight % is based on the weight of the copolymer and the plasticizer is diethyl sebacate.

2. The composition of claim 1, wherein the cationic (meth)acrylate copolymer (a) comprises 30 to 80 wt. % of radically polymerized C₁ to C₄ alkyl esters of acrylic or methacrylic acid and 70 to 20 wt. % (meth)acrylate monomers with a tertiary ammonium group in the alkyl radical.

3. The composition of claim 1, wherein the cationic (meth)acrylate copolymer (a) comprises 85 to 98 wt. % of radically polymerized C₁ to C₄ alkyl esters of acrylic or methacrylic acid and 15 to 2 wt. % (meth)acrylate monomers with a quaternary ammonium group in the alkyl radical.

4. The composition of claim 1, wherein the organic dicarboxylic or tricarboxylic acid (b) is selected from the group consisting of succinic acid (succinate), fumaric acid, citric acid, and mixtures thereof.

5. The composition of claim 1, wherein the acrylate or (meth)acrylate polymer (b) comprising an acid group is a copolymer of 30 to 70 wt. % ethyl acrylate or methyl methacrylate and 70 to 30 wt. % methacrylic acid.

6. The composition of claim 1, wherein the acrylate or (meth)acrylate polymer (b) comprising an acid group is polyacrylic acid.

7. The composition of claim 1, wherein the cationic radicals of the cationic (meth)acrylate copolymer (a) are neutralized by 2 to 50% by component (b).

8. The composition of claim 1, wherein a pharmaceutically active substance selected from the group consisting of analgesics, antirheumatics, anti-allergy substances, anti-arrhythmics, beta receptor blockers, calcium channel blockers, inhibitors of the renin/angiotensin system, broncholytics/anti-asthmatics, cholinergics, diuretics, substances that promote circulation, gout medications, flu medications, coronary medications, lipidlowering substances, gastrointestinal medications, psychoactive drugs, thrombocyte aggregation inhibitors, urologics, vein therapy medications, vitamins and minerals, is present.

9. The composition of claim 1, wherein a pharmaceutically active substance selected from the group consisting of morphine, derivatives of morphine, tramadol, acetylsalicylic acid, diclofenac, indometacin, lonazolac, ibuprofen, ketoprofen, propyphenazone, naproxen, paracetamol, flurbiprofen, dimetindene, quinidine, metoprolol, propranolol, oxprenolol, pindolol, atenolol, metoprolol, disopyramide, verapamil, diltiazem, gallopamil, nifedipine, nicardipine, nisoldipine, nimodipine, amiodipine, theophylline, salbutamol, sildenafil, terbutaline, ambroxol, aminophylline, choline theophyllinate, pyridostigmine, piretanide, furosemide, pentoxyfylline, naftidrofuryl, buflomedil, xantinol nicotinate, bencyclane, allopurinol, norephedrine, chlorphenamine, isosorbide mononitrate, isosorbide dinitrate, glycerin trinitrate, molsidomine, bezafibrate, fenofibrate, gemfibrozil, cerivastatin, pravastatin, fluvastatin, lovastatin, atorvastatin, simvastatin, xantinol, metoclopramide, amitryptiline, dibenzepine, venlafaxin, thioridazine, oxazepam, lithium, nitrofurantoin, dried plant extract, ascorbic acid, pharmaceutically acceptable salts thereof and mixtures thereof, is present.

10. (Amended) The composition of claim 1, wherein a pharmaceutically active substance selected from the group consisting of nicotine, glycerin trinitrate, scopolamine, clonidine, fentanyl, estradiol, testosterone, oxibutynine, diclophenac, deoxyribonucleic acids, ibuprofen, ketoprofen, diltiazem, propranolol, albuterol, alprazolam, amethocaine, atenolol, benzoporphyrin, buprenorphine, calcitonin, dithranol, diphencyprone, skin-penetrating peptides, peptides absorbed through the skin, eptazocine, ethinyl estradiol, methotrexate, naloxon and mixtures thereof, is present.

11. A method for producing the composition of claim 1, comprising
mixing components (a), (b) and (c), and optionally (d), with or without the addition of water, and
forming the composition by melting, injection-molding, casting, brushing, spraying, or pressing.

12. Cancelled

13. A composition comprising
(a) a (meth)acrylate copolymer comprising radically polymerized C₁ to C₄ alkyl esters of acrylic or methacrylic acid and (meth)acrylate monomers with a cationic ammonium group in the alkyl radical,
(b) 0.1 to 45 wt. % of an organic dicarboxylic or tricarboxylic acid or an acrylate or (meth)acrylate polymer or copolymer comprising an acid group,
(c) 20 to 80 wt. % of a plasticizer, and
(d) optionally, a pharmaceutically active substance, a pharmaceutically acceptable additive or mixture thereof,
wherein weight % is based on the weight of the copolymer (a) and the plasticizer (c) is dibutyl sebacate, diethyl sebacate or a mixture thereof, and
wherein the plasticizer is present in an amount of at least 90% of an original amount after storage for 6 months at 40° C. and 75% relative humidity.

14. The composition of claim 11, wherein the pharmaceutically active substance is present and wherein the pharmaceutically active substance is a vaccine.
